(19) United States
(12) Patent Application Publication
(10) Pub. No.: US 2015/0157244 A1
(43) Pub. Date: Jun. 11, 2015

(54) Jaundice Meter

(71) Applicant: KONICA MINOLTA, INC., Chiyoda-ku, Tokyo (JP)

(72) Inventors: Masanobu Kameda, Takaishi-shi (JP); Norihiro Tateda, Sakai-shi (JP); Masaharu Kanazawa, Suita-shi (JP); Takehiro Yashiro, Hirakata-shi (JP)

(73) Assignee: KONICA MINOLTA, INC., chiyoda-ku (JP)

(21) Appl. No.: 14/412,245

(22) PCT Filed: Jun. 17, 2013
(86) PCT No.: PCT/JP2013/003771

§ 371 Date: Dec. 31, 2014

(30) Foreign Application Priority Data
Jul. 2, 2012 (JP) 2012-148417

(51) Int. Cl. A61B 5/1495; A61B 19/00; A61B 5/00; A61B 5/145; A61B 5/1455

(52) U.S. Cl. CPC A61B 5/1495; A61B 5/14546; A61B 5/1455; A61B 5/443; A61B 19/00; A61B 5/7475; A61B 2019/4836

(57) ABSTRACT

Disclosed is a jaundice meter which comprises a jaundice measurement unit configured to measure a degree of jaundice by using light, and a warning control unit configured to recommend an inspection of the jaundice measurement unit. This jaundice meter has the warning control unit, so that it becomes possible to warn of an inspection timing to inspect a state of the jaundice measurement unit.

### TECHNICAL FIELD

The present invention relates to a jaundice meter, and more particularly to a jaundice meter having a function of warning of an inspection timing to inspect a state of the jaundice meter.

### BACKGROUND ART

Jaundice is a symptom emerging as a result of a phenomenon that, in a condition where there is a high level of bilirubin in blood, i.e., hyperbilirubinemia, caused when bilirubin generated from breakdown of hemoglobin in red blood cells is not excreted from a living body due to some kind of disorder, the high-level bilirubin in the blood is deposited in a living tissue, thereby causing yellowish pigmentation of the living tissue. Bilirubin has a high affinity for fibroelastic tissue, so that it is deposited in a skin, a sclera, a blood vessel and the like abundant in fibroelastic tissue. A fetus in a prenatal period has a red blood cell count about 1.5 to 2 times greater than that of an adult human, in order to compensate for deficiency of oxygen due to poor efficiency of oxygen exchange by placenta, as compared to oxygen exchange by lungs. When lung breathing becomes possible after birth, the number of red blood cells in blood becomes excess, and thus an excess part of the red blood cells are broken down. As a result of the above physiological phenomenon, a newborn infant becomes jaundiced. This jaundice is referred to as “neonatal jaundice”.

In some cases, the neonatal jaundice becomes serious disease, and involves a risk of exerting a critical influence on life and brain development of a newborn infant. For this reason, it is necessary to early and appropriately detect and treat the neonatal jaundice. From the standpoint of accurate determination on an intensity of jaundice symptoms (degree (of severity) of jaundice), a bilirubin level in blood should be measured. However, it is difficult, and not realistic, to collect blood from each newborn infant to measure a bilirubin level in the blood.

Therefore, a jaundice meter for measuring a degree of jaundice has heretofore been developed, and an example thereof is disclosed in the following Patent Literatures 1 to 3. Each jaundice meter disclosed in the Patent Literatures 1 to 3 is an apparatus designed to measure a degree of jaundice based on a phenomenon that a light component on a short wavelength side is absorbed by bilirubin deposited in a subcutaneous tissue in a greater amount as compared to a light component on a long wavelength side. More specifically, in each jaundice meter disclosed in these Patent Literatures, the Lambert-Beer law is utilized, wherein, first of all, light is emitted from the jaundice meter to enter a skin. The entering light is scattered by a fatty layer of the subcutaneous tissue having bilirubin deposited therein and others, and the resulting backwardly scattered light is emitted from a surface of the skin and received by the jaundice meter. The jaundice meter is operable, based on the received backwardly scattered light, to derive two light intensities at a first wavelength having a relatively large bilirubin absorption coefficient and a second wavelength having a relatively small bilirubin absorption coefficient, and a concentration of the bilirubin deposited in the subcutaneous tissue is measured from a difference between the light intensities. Such an optical jaundice meter has an advantage of being able to obtain a measurement result in a non-invasive manner and within a relatively short period of time.

Meanwhile, a jaundice meter is a medical device, and therefore should be used in a normal state in order to avoid wrong diagnosis. For this reason, in jaundice meters, with a view to obtaining a normal measurement result, it is a recommended to perform measurement after inspecting a state of the meter. Particularly, the above optical jaundice meter is configured to utilize backwardly scattered light. Thus, in a situation where a light intensity of the meter becomes lower than a desired value due to degradation of a light source, or in a situation where the light intensity becomes lower than a desired value due to damage to a light guide member (e.g., optical fibers or the like) for guiding light from a light source to a measurement probe, the light intensity becomes insufficient, causing difficulty in obtaining a normal measurement result.

An instruction manual of the meter clearly says that measurement should be performed after an inspection about whether the meter is in a normal state. However, in actual usage sites, such a caution is not recognized by a user, in some cases. Even when recognized, the user is likely to fail to judge at what timing (intervals) the inspection should be performed. Moreover, in a situation where a plurality of users share one jaundice meter, it is unclear whether or not a previous user performed the inspection, and thereby it is unable to judge whether or not the inspection should be performed.

### CITATION LIST

### Patent Literature

Patent Literature 1: JP 62-2809 B

Patent Literature 2: JP 04-127035 A

Patent Literature 3: JP 2012-61232 A

### SUMMARY OF INVENTION

The present invention has been made in view of the above circumstances, and an object thereof is to provide a jaundice meter capable of warning of an inspection timing to inspect a state of the meter.

The jaundice meter of the present invention comprises a jaundice measurement unit configured to measure a degree of jaundice by using light, and a warning control unit configured to recommend an inspection of the jaundice measurement unit. The jaundice meter of the present invention has the warning control unit. This makes it possible to warn of an inspection timing to inspect a state of the jaundice measurement unit.

These and other objects, features, and advantages of the present invention will become apparent upon reading of the following detailed description along with the accompanying drawings.

### BRIEF DESCRIPTION OF DRAWINGS

FIG. 1 is views illustrating an external configuration of a jaundice meter according to one embodiment of the present invention.

FIG. 2 is a perspective view illustrating a stand for allowing the jaundice meter according to the embodiment to be placed thereon.

FIG. 3 is a perspective view illustrating a state in which the jaundice meter according to the embodiment is placed on the stand.

FIG. 4 is a block diagram illustrating a configuration of the jaundice meter according to the embodiment.

FIG. 5 is a flowchart illustrating an inspection operation of the jaundice meter according to the embodiment.

FIG. 6 is a flowchart illustrating a calibration operation of the jaundice meter according to the embodiment.

FIG. 7 is diagrams illustrating a display screens displayed on an output unit of the jaundice meter according to the embodiment.

### DESCRIPTION OF EMBODIMENTS

With reference to the drawings, one embodiment of the present invention will now be described. In the figures, elements or components assigned with the same reference numeral or sign mean that they are the same elements or components, and their duplicated description will be appropriately omitted. In this specification, a collective term is designated by a reference sign without any suffix, and a term representing an individual element or component is designated by a reference sign with a suffix.

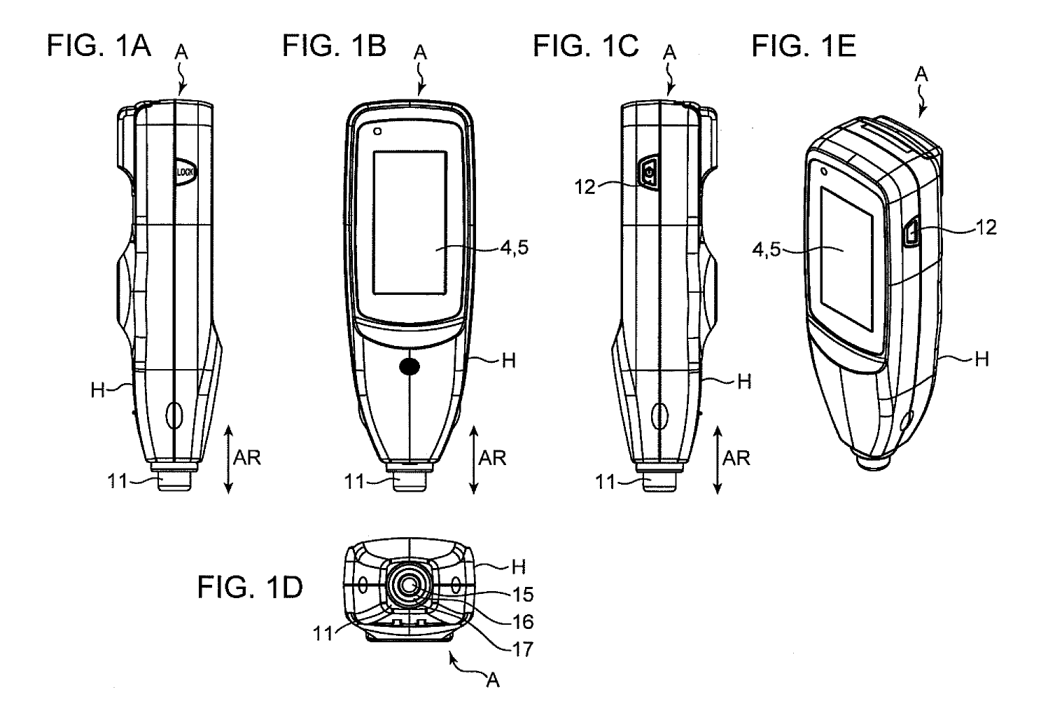

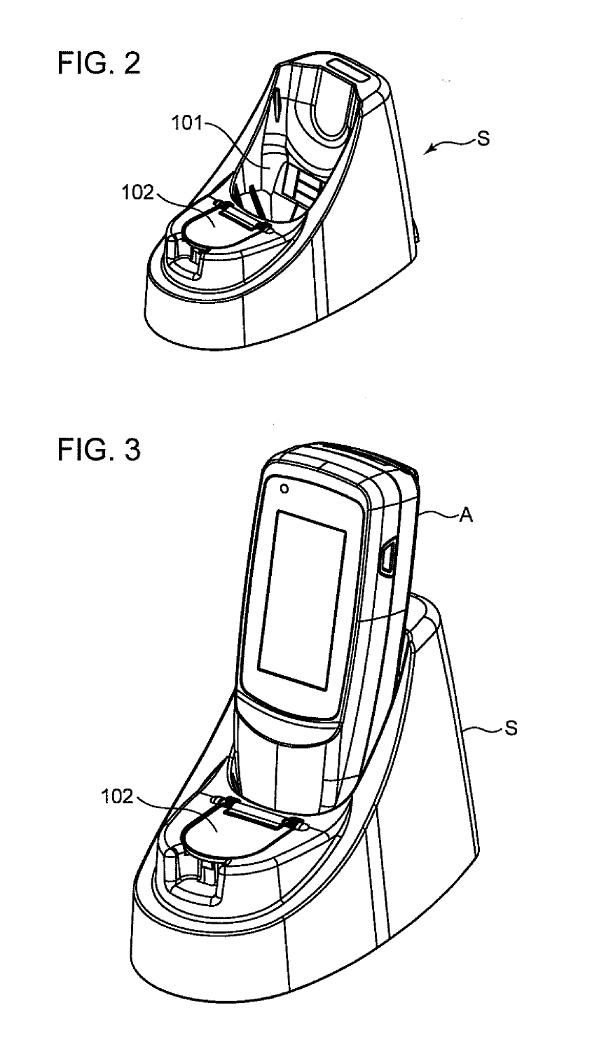

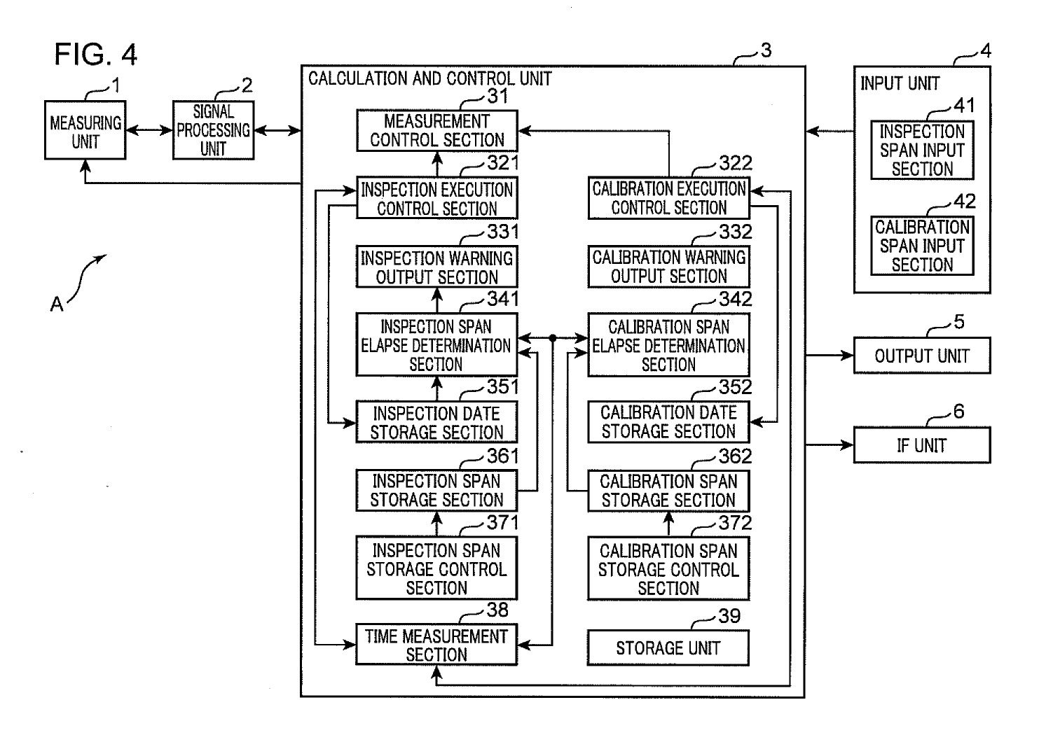

FIG. 1 is views illustrating an external configuration of a jaundice meter according to one embodiment of the present invention. FIG. 1A, FIG. 1B, FIG. 1C, FIG. 1D and FIG. 1E are, respectively, a first side view (left side view), a front view, a second side view (right side view), a view when viewed from an entrance-exit port (nose edge), and a perspective front view. FIG. 2 is a perspective view illustrating a stand for allowing the jaundice meter according to this embodiment to be placed thereon. FIG. 3 is a perspective view illustrating a state in which the jaundice meter according to this embodiment is placed on the stand. FIG. 4 is a block diagram illustrating a configuration of the jaundice meter according to this embodiment.

The jaundice meter according to this embodiment is an optical apparatus designed to measure a degree of jaundice based on a phenomenon that a light component on a short wavelength side is absorbed by bilirubin deposited in a subcutaneous tissue in a greater amount as compared to a light component on a long wavelength side, wherein the jaundice meter comprises: a jaundice measurement unit configured to measure the degree of jaundice; a warning control unit configured to recommend an inspection of the jaundice measurement unit; and a housing which houses the jaundice measurement unit and the warning control unit.

As used in this specification, the term “inspection” means to check whether or not a jaundice meter is in a normal state, or check whether or not an impediment to a normal measurement occurs in the jaundice meter (presence or absence of an impediment), and the term “calibration” means to correct an error in a measurement value measured by a jaundice meter in such a manner as to allow the measurement value to become equal to or closer to a true measurement value.

More specifically, as illustrated in FIG. 1, the jaundice meter A is a handheld apparatus, and comprises a casing (housing, main frame) H having a size capable of being fitted into a hand of a user (a measurer, a medical staff such as a medical doctor or a nurse, or the like). The casing H is an approximately rectangular parallelepiped-shaped box-like member having one end (nose end) which is tapered toward an edge thereof. As illustrated in FIG. 1B, an aftermentioned input unit 4 and an aftermentioned output unit 5 are provided in a region of one principal surface of the casing H on the side of the other end (in a base end region of the front surface). Further, a circular cylindrical-shaped protruding member 11 is provided on the side of the head end in such a manner as to be extendable and retractable with respect to the casing H, as indicated by the double arrow AR. The protruding member 11 is biased against the casing H in a protruding direction (a downward direction of the double arrow AR) by a non-illustrated biasing device (biasing member) such as a spring member, and is configured to be pushed into the casing H against a biasing force of the biasing device when a user presses the protruding member 11 onto a part (e.g., a forehead region or a chest region) of a living body of a person to be measured (measurement target), to thereby cause a light source of an aftermentioned measuring unit 1 to produce light. When the light source produces light, measurement light radiated from the light source exits an edge face of the protruding member 11, and enters a skin of the measurement target, and then backwardly scattered light in a subcutaneous tissue enters the aftermentioned measuring unit 1 inside the casing H, via the protruding member 11. That is, as illustrated in FIG. 1D, the edge face of the protruding member 11 is formed as an entrance-exit port serving as an exit port for emitting the measurement light therethrough, and an entrance port for introducing the scattered light therethrough. Further, as illustrated in FIG. 1C, a power on-off switch 12 for manually turning on and off the jaundice meter A is provided in a base end region of one side surface (right side surface) of the casing H.

Further, a stand (holder) S illustrated in FIG. 2 is prepared for the jaundice meter A having the above shape, as a holding place in a non-use state. This stand S has a shape obtained by obliquely cutting a long elliptic cylindrical shape, wherein an obliquely cut surface thereof is provided with a receiving concave portion 101 for receiving therein the jaundice meter A, and an inspection concave portion for receiving therein the protruding member 11 during inspection of a state of the jaundice measurement unit. A neutral density filter and a diffusing plate are arranged on a bottom wall of the inspection concave portion. Thus, when the protruding member 11 is inserted into the inspection concave portion to start the inspection, measurement light emitted from the protruding member 11 is reflected by the bottom wall of the inspection concave portion, while being reduced in its light intensity and avoiding mirror reflection, and introduced into the protruding member 11 as diffusion light. In the example illustrated in FIG. 2, an opening of the inspection concave portion is closed by a cap (cover member) 102. FIG. 3 illustrates a state in which the stand S is placed on the stand S while being fitted into the receiving concave portion 101 of the stand S. The stand S is fitted into the receiving concave portion 101 of the stand S, so that it becomes possible to hold the jaundice meter A while protecting the edge face of the protruding portion 11.

More specifically, as illustrated in FIG. 4, the jaundice measurement unit and the warning control unit in the jaundice meter A comprise: a measuring unit 1, a signal processing unit 2, a calculation and control unit 3, an input unit 4, an output unit 5, and an interface unit (hereinafter abbreviated as “IF unit”) 6.

The measuring unit 1 is a device configured to, under control of the calculation and control unit 3, emit light to enter a skin, and, after receiving backwardly scattered light which exits a surface of the skin as a result of scattering caused by a fatty layer of a subcutaneous tissue having bilirubin deposited therein, measure two light components of the received light having different wavelengths. An exit face 16 for emitting the measurement light therethrough is formed in an annular shape (ring shape) to face outwardly at the edge face of the protruding member 11, as illustrated in FIG. 1D, and an entrance face 15 for introducing therethrough scattered light which is returned light as a result of scattering of the emitted measurement light in a subcutaneous tissue is formed in a circular shape at a position inward of the annular-shaped (ring-shaped) exit face 16 to face outwardly at the edge face of the protruding member 11, as illustrated in FIG. 1D. Further, another entrance face 17 for introducing the scattered light therethrough is formed in an annular shape (ring shape) at a position outward of the annular-shaped (ring-shaped) exit face 16 to face outwardly at the edge face of the protruding member 11, as illustrated in FIG. 1D. That is, the exit face 16 and the entrance faces 15, 17 are concentrically located. Further, the measuring unit 1 is internally provided with a two types of optical filters (bandpass filters) each capable of filtering light introduced from the entrance faces 15, 17 to output a light component of a given transparent wavelength region. In this embodiment, the first optical filter is configured such that a center wavelength of the transparent wavelength region thereof becomes 450 nm so as to allow blue light to pass therethrough, and the second optical filter is configured such that a center wavelength of the transparent wavelength region thereof becomes 500 nm so as to allow green light to pass therethrough. The light component received through each of the above optical filters is photoelectrically converted to a current having a magnitude corresponding to an intensity thereof, and an obtained measurement result (received-light signals) is output to the signal processing unit 2.

The signal processing unit 2 is a circuit designed to subject the measurement result (received-light signals) obtained in the measuring unit 1 to a given signal processing, and output a result of the signal processing to the calculation and control unit 3. For example, the signal processing unit 2 comprises: an IV conversion section configured to convert each of the received-light signals from a current signal into a voltage signal; an amplification section configured to amplify an output of the IV conversion section at a predetermined given gain; and an AD conversion section configured to convert an output of the amplification section from an analog signal into a digital signal (to AD-convert an output of the amplification section). The AD conversion section is also configured to output a digital signal as a result of the conversion, to the calculation and control unit 3. That is, an output of the measuring unit 1 is input into the calculation and control unit 3 via the IV conversion sections, the amplification sections and the AD conversion sections.

The input unit 4 is a device designed to allow a variety of data associated with a jaundice measurement, e.g., various commands such as a command instructing the jaundice meter to start the jaundice measurement and a command instructing the jaundice meter to start calibration, and input data such as an identifier (ID) of a measurement target, to be input into the jaundice meter A therethrough. The output unit 5 is a device designed to output a command and data input through the input unit 4, and the degree of jaundice measured by the jaundice meter A, and composed, for example, of a display such as a CRT display, a liquid crystal display (LCD), an organic EL display or a plasma display, or a printing device such as a printer. In this embodiment, the input unit 4 is composed of a position input device, such as a resistive type or a capacitive type, configured to detect and input an operation position, and the output unit 5 is composed of a display, wherein a touch panel is made up of the position input device and the display.

In this touch panel, the position input device is provided on a display surface of the display, and the display is configured to display thereon one or more candidates for inputtable contents, wherein when a user touches one position where a content that the user wants to input is displayed, the position is detected by the position input device, and the content displayed at the detected position is input into the jaundice meter A as a user's operation input content. In such a touch panel, a user is more likely to intuitively understand an input operation, so that it becomes possible to provide a jaundice meter A which is easy for a user to operate or handle.

The IF unit 6 is a circuit designed to perform data input and output between an external device and the jaundice meter A, and composed, for example, of an interface circuit using USB (Universal Serial Bus) Standard.

The calculation and control unit 3 is designed to control each section of the jaundice meter A depending on a function of each section, so as to calculate the degree of jaundice. For example, the calculation and control unit 3 is composed of a micro-computer comprising: a CPU (Central Processing Unit); a non-volatile storage element, such as a RPM (Read Only Memory) or an EEPROM (Electrically Erasable Programmable Read Only Memory), preliminarily storing therein various programs to be executed by the CPU, data necessary for the execution and others; a volatile storage element, such as a RAM (Random Access Memory), serving as a so-called working memory for the CPU; and a peripheral circuit of the CPU. In the example illustrated in FIG. 4, the non-volatile storage element and the volatile storage element are depicted as an inspection date storage section 351, an inspection span storage section 361, a calibration date storage section 352, a calibration span storage section 362 and a storage unit 39. In the calculation and control unit 3, as a result of executing the programs, a measurement control section 31, an inspection execution control section 321, an inspection warning output section 331, an inspection span elapse determination section 341, an inspection span storage control section 371, a calibration execution control section 322, a calibration warning output section 332, a calibration span elapse determination section 342, a calibration span storage control section 372 and a time measurement section 38 are functionally formed.

The inspection date storage section 351 is designed to store therein inspection date T when a state of the jaundice measurement unit, i.e., a state of the measuring unit 1 and the signal processing unit 2, is inspected. The inspection date storage section 351 may be configured to store therein only most recent inspection date T1, or may be configured to store therein a plurality of past inspection dates Tn including most recent inspection date T1, so as to record an inspection history.

The inspection span storage section 361 is designed to store therein an inspection span T0 which is a time interval (time span) at which the inspection is to be performed. The inspection span storage section 361 may be configured to store therein one inspection span T0, for example, to store therein a default value T0d of the inspection span T0 at the time of manufacturing or shipment, and, when a user sets a value of the inspection span T0, overwrite the default value T0d with the set value. Alternatively, the inspection span storage section 361 may be configured to store therein a plurality of inspection spans T0n, for example, to store therein a default value T0d of the inspection span T0 at the time of manufacturing or shipment, and a user setup value T0y of the inspection span T0 set by a user. In order to allow input of a user setup inspection span T0y, the input unit 4 comprises an inspection span input section 41 configured to allow the inspection span T0 to be externally input therethrough. The default value T0d of the inspection span T0 is appropriately set, for example, to half-day, or one day, or two days.

The calibration date storage section 352 is designed to store therein calibration date D when the jaundice measurement unit, i.e., the measuring unit 1 and the signal processing unit 2, is calibrated. The calibration date storage section 352 may be configured to store therein only most recent calibration date D1, or may be configured to store therein a plurality of past calibration dates Dn including most recent calibration date, so as to record an calibration history.

The calibration span storage section 362 is designed to store therein a calibration span D0 which is a time interval (time span) at which the calibration is to be performed. The calibration span storage section 362 may be configured to store therein one calibration span T0, for example, to store therein a default value D0d of the calibration span D0 at the time of manufacturing or shipment, and, when a user (or manufacturer or serviceperson) sets a value of the calibration span D0, overwrite the default value D0d with the set value. Alternatively, the calibration span storage section 362 may be configured to store therein a plurality of calibration spans D0n, for example, to store therein a default value D0d of the calibration span D0 at the time of production or shipment, and a user setup value D0y of the inspection span D0 set by a user. In order to allow input of a user setup calibration span D0y, the input unit 4 comprises an calibration span input section 42 configured to allow the calibration span D0 to be externally input therethrough. The default value D0d of the calibration span D0 is appropriately set, for example, to half-year or one year.

The storage unit 39 is designed to store therein various programs, such as a jaundice calculation program for calculating the degree of jaundice from a measurement result in the measuring unit 1, a program for controlling each section of the jaundice meter A, an inspection warning program for warning of the inspection, and a calibration warning program for warning of the calibration, and various data, such as measurement results. The storage unit 39 also serves as a working memory, as mentioned above.

The time measurement section 38 is designed to measure time and date. In this embodiment, determination on elapse of the inspection span T0 and determination on elapse of the calibration span D0 are performed by day. Thus, the time measurement section 38 may be configured to measure at least day, or may be configured to measure time and day, or day, month and year, or time, day, month and year. Then, the time measurement section 38 is operable, in response to a request from the inspection execution control section 321, to notify the inspection execution control section 321 of a time and date at the time of the request. The time measurement section 38 is operable, in response to a request from the inspection span elapse determination section 341, to notify the inspection span elapse determination section 341 of a time and date at the time of the request. The time measurement section 38 is operable, in response to a request from the calibration execution control section 322, to notify the calibration execution control section 322 of a time and date at the time of the request. The time measurement section 38 is operable, in response to a request from the calibration span elapse determination section 342, to notify the calibration span elapse determination section 342 of a time and date at the time of the request.

The measurement control section 31 is designed to control the measuring unit 1 to thereby measure the degree of jaundice. The measuring unit 1 is operable, under control of the measurement control section 31, to emit white light as measurement light to enter a skin, and receive backwardly scattered light which exits a surface of the skin as a result of scattering caused by a fatty layer of a subcutaneous tissue to measure a plurality of light components having different wavelengths, and then to output an obtained measurement result to the measurement control section 31 via the signal processing unit 2, as mentioned above. The measurement control section 31 is operable, based on the measurement result, to calculate the degree of jaundice, and output the measurement result to the output unit 5. When the inspection and calibration are executed under control of the inspection execution control section 321 and the calibration execution control section 322, respectively, the measurement control section 31 operates in the same manner as that during measurement of the degree of jaundice, to perform the inspection and calibration.

The inspection execution control section 321 is designed to perform inspection of a state of the jaundice measurement unit, i.e., in this embodiment, inspection of the measuring unit 1 and the signal processing unit 2. More specifically, the inspection execution control section 321 is operable, in response to receiving a user's instruction for start of inspection from the input section 4, to cause the measurement control section 31 to perform the same operation as that during measurement of the degree of jaundice. Then, the inspection execution control section 321 is operable, in response to receiving a notification of a time and date from the time measurement section 38, to cause the notified time and date to be stored in the inspection date storage section 351 as an inspection date T when the inspection is performed. In this case, the inspection execution control section 321 may be configured to instruct the time measurement section 38 to store the inspection date T in the inspection date storage section 351.

The inspection span elapse determination section 341 is designed to, at a preset appropriate given timing, e.g., the time of activation of the jaundice meter A, determine whether or not the inspection span T0 stored in the inspection span storage section 361 has elapsed from most recent inspection date T1 stored in the inspection date storage section 351. As a result of the determination, when the inspection span T0 has elapsed from the most recent inspection date T1 at the timing of the determination, it is judged that it is necessary to warn of the inspection, and thereby the inspection span elapse determination section 341 is operable to notify the inspection warning output section 331 of an instruction for execution of the inspection warning, so as to perform the inspection warning. On the other hand, as a result of the above determination, when the inspection span T0 has not elapsed from the most recent inspection date T1 at the timing of the determination, it is judged that it is not necessary to warn of the inspection, and thereby the inspection span elapse determination section 341 terminates the inspection warning processing routine.

The inspection warning output section 331 is designed to output the inspection warming to recommend inspection of the jaundice measurement unit i.e., in this embodiment, inspection of the measuring unit 1 and the signal processing unit 2, to the output unit 5, when the inspection span elapse determination section 341 determines that the inspection span T0 has elapsed, and the inspection warning output section 331 is notified of the instruction for execution of the inspection warning, from the inspection span elapse determination section 341.

The inspection span storage control section 371 is designed to cause a user setup inspection span T0y input through the inspection span input section 41 to be stored in the inspection span storage section 361.

The calibration execution control section 322 is designed to perform calibration of the jaundice measurement unit i.e., in this embodiment, calibration of the measuring unit 1 and the signal processing unit 2. More specifically, the calibration execution control section 322 is operable, after the jaundice meter A is set to measure a calibration plate, and in response to receiving a user's instruction for start of calibration from the input section 4, to cause the measurement control section 31 to perform the same operation as that during measurement of the degree of jaundice. Then, the calibration execution control section 322 is operable to calculate a calibration value for correcting a deviation between a result of this measurement and a pre-stored proper measurement result to be obtained from measurement of the calibration plate, and set the calculated calibration value to the measurement control section 31. Then, the calibration execution control section 322 is operable, in response to receiving a notification of a time and date from the time measurement section 38, to cause the notified time and date to be stored in the calibration date storage section 352 as a calibration date D when the calibration is performed. In this case, the calibration execution control section 322 may be configured to instruct the time measurement section 38 to store the calibration date D in the calibration date storage section 352.

The calibration span elapse determination section 342 is designed to, at a preset appropriate given timing, e.g., the time of activation of the jaundice meter A, determine whether or not the calibration span D0 stored in the calibration span storage section 362 has elapsed from most recent calibration date D1 stored in the calibration date storage section 352. As a result of the determination, when the calibration span D0 has elapsed from the most recent calibration date D1 at the timing of the determination, it is judged that it is necessary to warn of the calibration, and thereby the calibration span elapse determination section 342 is operable to notify the calibration warning output section 332 of an instruction for execution of the calibration warning, so as to perform the calibration warning. On the other hand, as a result of the above determination, when the calibration span D0 has not elapsed from the most recent calibration date D1 at the timing of the determination, it is judged that it is not necessary to warn of the calibration, and thereby the calibration span elapse determination section 342 terminates the calibration warning processing routine.

The calibration warning output section 332 is designed to output the calibration warming to recommend calibration of the jaundice measurement unit, i.e., in this embodiment, calibration of the measuring unit 1 and the signal processing unit 2, to the output unit 5, when the calibration span elapse determination section 342 determines that the calibration span D0 has elapsed, and the calibration warning output section 332 is notified of the instruction for execution of the calibration warning, from the calibration span elapse determination section 342.

The calibration span storage control section 372 is designed to cause a user setup calibration span D0y input through the calibration span input section 42 to be stored in the calibration span storage section 362.

In the jaundice meter A according to this embodiment, a combination of the measuring unit 1 and the signal processing unit 2 is equivalent to one example of the jaundice measurement unit, and a combination of the inspection warning output section 331, the inspection span elapse determination section 341, the inspection date storage section 351, the inspection span storage section 361, the calibration warning output section 332, the calibration span elapse determination section 342, the calibration date storage section 352, the calibration span storage section 362 and the time measurement section 38 is equivalent to one example of the warning control unit.

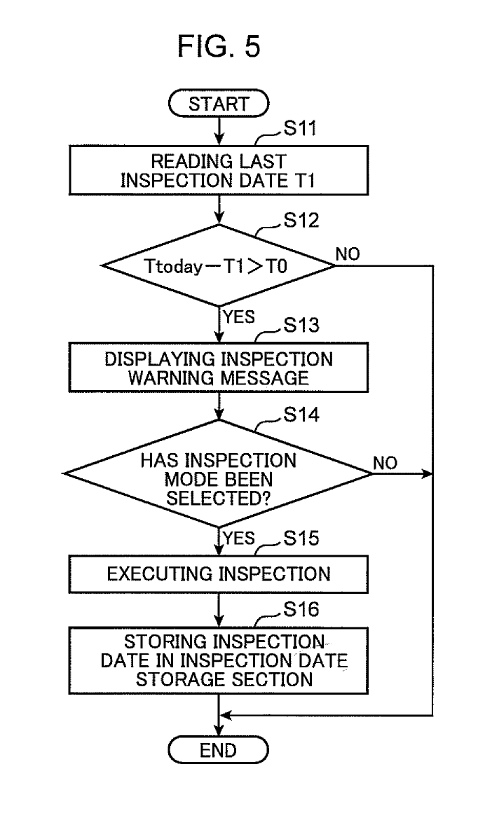

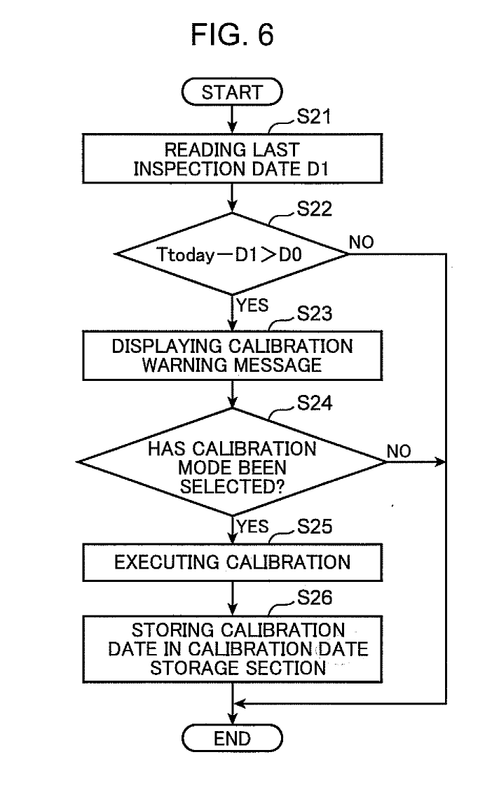

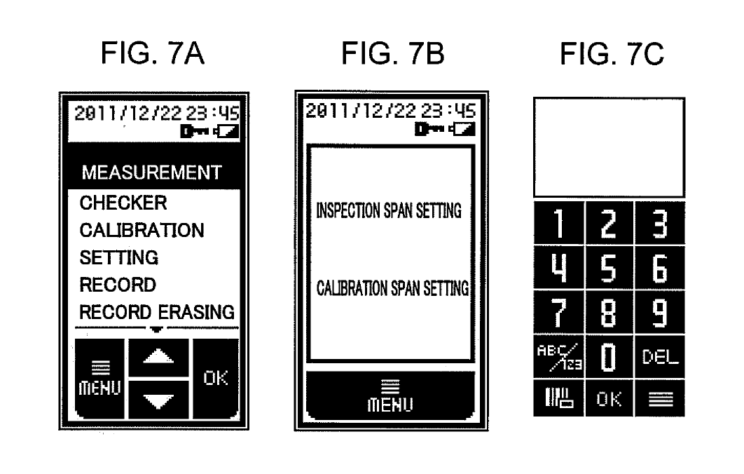

Next, an inspection warning operation for performing the inspection warning and a calibration warning operation for performing the calibration warning, in the jaundice meter A, will be described. FIG. 5 is a flowchart illustrating the inspection operation in the jaundice meter according to this embodiment. FIG. 6 is a flowchart illustrating the calibration operation in the jaundice meter according to this embodiment. FIG. 7 is diagrams illustrating a display screens displayed on the output unit of the jaundice meter according to this embodiment. FIG. 7A, FIG. 7B and FIG. 7C illustrate a main menu display screen, a sub-menu display screen, and a numerical key display screen, respectively.

First, the inspection warning operation in the jaundice meter A will be described. For example, at a given timing to determine whether or not the inspection warning is performed, e.g., at a time when the jaundice meter A is activated by operating the power on-off switch 12 of the jaundice meter A, the jaundice meter A first determines whether or not the inspection span T0 has elapsed from the most recent inspection date T1. As a result of the determination, when the inspection span T0 has elapsed, the jaundice meter A operates to perform the inspection warning to recommend the inspection, and, when the inspection span T0 has not elapsed, the processing routine is terminated.

More specifically, as illustrated in FIG. 5, the inspection span elapse determination section 341 issues a request of a present time and date to the time measurement section 38, and receives a notification of the present time and date from the time measurement section 38. The inspection span elapse determination section 341 also issues a request of an inspection span T0 to the inspection span storage section 361, and receives a notification of the inspection span T0 from the inspection span storage section 361. Further, the inspection span elapse determination section 341 issues a request of a most recent inspection date T1 to the inspection date storage section 351, and receives a notification of the most recent inspection date T1 from the inspection date storage section 351 (S11).

Then, in units of day, the inspection span elapse determination section 341 subtracts the most recent inspection date T1 notified from the inspection date storage section 351, from the present date Ttoday notified from the time measurement section 38, and determines whether or not a result of the subtraction (Ttoday−T1) is greater than the inspection span T0 notified from the inspection span storage section 361 (S12).

As a result of this determination, when the subtraction result is greater than the inspection span T0 (YES: Ttoday−T1>T0), it is judged that the inspection warning is necessary, and the inspection span elapse determination section 341 notifies the inspection warning output section 331 of an instruction for execution of the inspection warning, and processing S13 is executed. On the other hand, as a result of the above determination, when the subtraction result is equal to or less than the inspection span T0 (NO: Ttoday−T1≦T0), it is judged that the inspection warning is not necessary, and the processing routine is terminated.

When the inspection warning output section 331 is notified of the instruction for execution of the inspection warning, from the inspection span elapse determination section 341, the inspection warning output section 331 outputs the inspection warning to recommend inspection of a state of the jaundice measurement unit i.e., in this embodiment, inspection of the measuring unit 1 and the signal processing unit 2, in processing S13. For example, this inspection warning is an inspection warning message such as “Please perform inspection”.

A user can judge the necessity of the inspection by watching the inspection warning message. When performing the inspection, a user inserts the protruding member 11 of the jaundice meter A into the inspection concave portion of the stand S. Then, the user inputs an instruction for start of the inspection into the jaundice meter A through the input unit 4.

This input of an instruction for start of the inspection is performed using a touch panel composed of the input unit 4 and the output unit 5. For example, on this touch panel, the main menu illustrated in FIG. 7A is displayed to input an instruction fort an operation mode of the jaundice meter A so as to operate the jaundice meter A in the operation mode. This main menu includes: a “measurement” field for inputting, into the jaundice meter A, an instruction for measurement of the degree of jaundice so as to operate the jaundice meter A in a measurement mode during which the degree of jaundice is measured; a “checker” field for inputting, into the jaundice meter A, an instruction for execution of the inspection of the jaundice meter A so as to operate the jaundice meter A in an inspection mode during which the inspection is performed; a “calibration” field for inputting, into the jaundice meter A, an instruction for execution of the calibration so as to operate the jaundice meter A in a calibration mode; a “setting” field for inputting, into the jaundice meter A, an instruction for various settings such as setting of the inspection span and setting of the calibration span, so as to operate the jaundice meter A in a setting mode during which the setting is permitted; a “record” field for inputting, into the jaundice meter A, an instruction for display of past measurement results so as to operate the jaundice meter A in a record mode during which the past measurement results are displayed; and a “record erasing” field for inputting, into the jaundice meter A, an instruction for erasing of past measurement results so as to operate the jaundice meter A in a record erasing mode during which the past measurement results are erased. A user touches the “checker” field displayed on the main menu input screen displayed on the touch panel to thereby input an instruction for start of the measurement, into the jaundice meter A.

In the jaundice meter A, the inspection execution control section 321 determines whether or not the inspection mode has been selected through the touch panel composed of the input section 4 and the output section 5 (S14). In the example illustrated in FIG. 7A, the inspection execution control section 321 determines whether or not the “checker” field in the touch panel is touched. As a result of the determination, when it is determined that the inspection mode has been selected, the jaundice meter A is operated in the inspection mode, wherein, as execution of the inspection, the inspection execution control section 321 operates to cause the measurement control section 31 to perform the same operation as that during measurement of the degree of jaundice, and the measurement control section 31 operates to cause a result of the measurement to be output to the output unit 5 (S15). In this way, the inspection is executed. The user checks the measurement result output to the output unit 5, and, when the measurement result has almost the same value as that of a normally obtained measurement result, determines that the jaundice meter A is in a normal state. On the other hand, when the value of the measurement result is not almost the same value as that of the normally obtained measurement result, determines that the jaundice meter A is not in a normal state. Then, the inspection execution control section 321 receive a notification of a time and date from the time measurement section 38, and operates to cause the notified time and date to be stored in the inspection date storage section 351 as an inspection date T when the inspection is performed (S16), and the processing routine is terminated.

As a result of the above inspection, when the jaundice meter A is determined to be not in a normal state, it is necessary to correct impediment in the jaundice meter A. For example, it is conceivable to implement calibration and/or countermeasures against degradation of a light source and damage of a light guide member. Further, in a situation where no calibration is performed for a long period of time, even when the jaundice meter A is determined to be in a normal state as a result of the above inspection, a deviation of measurement value is likely to occur due to aging or the like. Thus, when a given time has elapsed, the calibration of the jaundice meter A needs to be performed to correct such a deviation of measurement value.

Next, the calibration warning operation in the jaundice meter A will be described. For example, at a given timing to determine whether or not the calibration warning is performed, e.g., at a time when the jaundice meter A is activated by operating the power on-off switch 12 thereof, the jaundice meter A first determines whether or not the calibration span D0 has elapsed from the most recent calibration date D1. As a result of the determination, when the calibration span D0 has elapsed, the jaundice meter A operates to perform the calibration warning to recommend the calibration, and, when the calibration span D0 has not elapsed, and the processing routine is terminated.

More specifically, as illustrated in FIG. 6, the calibration span elapse determination section 342 issues a request of a present time and date to the time measurement section 38, and receives a notification of the present time and date from the time measurement section 38. The calibration span elapse determination section 342 also issues a request of a calibration span D0 to the calibration span storage section 362, and receives a notification of the calibration span D0 from the calibration span storage section 362. Further, the calibration span elapse determination section 342 issues a request of a most recent calibration date D1 to the calibration date storage section 352, and receives a notification of the most recent calibration date D1 from the calibration date storage section 352 (S21).

Then, in units of day, the calibration span elapse determination section 342 subtracts the most recent calibration date D1 notified from the calibration date storage section 352, from the present date Ttoday notified from the time measurement section 38, and determines whether or not a result of the subtraction (Ttoday−D1) is greater than the calibration span D0 notified from the calibration span storage section 362 (S22).

As a result of this determination, when the subtraction result is greater than the calibration span D0 (YES: Ttoday−D1>D0), it is judged that the calibration warning is necessary, and the calibration span elapse determination section 342 notifies the calibration warning output section 332 of an instruction for execution of the calibration warning, and processing S23 is executed. On the other hand, as a result of the above determination, when the subtraction result is equal to or less than the calibration span D0 (NO: Ttoday−D1≦D0), it is judged that the calibration warning is not necessary, and the processing routine is terminated.

When the calibration warning output section 332 is notified of the instruction for execution of the calibration warning, from the calibration span elapse determination section 342, the calibration warning output section 332 outputs the calibration warning to recommend calibration of the jaundice measurement unit i.e., in this embodiment, calibration of the measuring unit 1 and the signal processing unit 2, in processing S23. For example, this calibration warning is a calibration warning message such as “Please perform calibration”.

A user can judge the necessity of the calibration by watching the calibration warning message. The calibration is performed by measuring a calibration plate using a calibration jig.

In the jaundice meter A, the calibration execution control section 322 determines whether or not the calibration mode has been selected through the touch panel composed of the input section 4 and the output section 5 (S24). In the example illustrated in FIG. 7A, the calibration execution control section 322 determines whether or not the “calibration” field in the touch panel is touched. As a result of the determination, when it is determined that the calibration mode has been selected, the jaundice meter A is operated in the calibration mode, wherein the calibration execution control section 322 operates to cause the measurement control section 31 to perform the same operation as that during measurement of the degree of jaundice, and the measurement control section 31 operates to execute the calibration based on a result of the measurement (S25). Then, the calibration execution control section 322 receive a notification of a time and date from the time measurement section 38, and operates to cause the notified time and date to be stored in the calibration date storage section 352 as a calibration date D when the calibration is performed (S26), and the processing routine is terminated.

In the above embodiment, when a user wants to set the inspection span T0, an operation therefor is performed, for example, in the following manner. In the example illustrated in FIG. 7A, a user touches the “setting” field in the main menu displayed on the touch panel, so that an instruction for start of the setting is input into the jaundice meter A, and thereby the jaundice meter A is operated in the setting mode.

In the setting mode, the inspection span storage control section 371 operates to cause a span setting sub-menu illustrated in FIG. 7B for inputting, into the jaundice meter A, an instruction for a span setting mode of the jaundice meter A to operate the jaundice meter A in the span setting mode, to be displayed on the touch panel. This span setting sub-menu includes: an “inspection span setting” field for inputting, into the jaundice meter A, an instruction for execution of setting of the inspection span T0 to operate the jaundice meter A in an inspection span setting mode during which the inspection span T0 is set; and a “calibration span setting” field for inputting, into the jaundice meter A, an instruction for execution of setting of the calibration span D0 to operate the jaundice meter A in a calibration span setting mode during which the calibration span D0 is set. The user touches the “inspection span setting” field in the span setting sub-menu displayed on the touch panel, so that an instruction for inspection span setting is input into the jaundice meter A, and thereby the jaundice meter A is operated in the inspection span setting mode.

In the inspection span setting mode, the inspection span storage control section 371 operates to cause a numerical key set illustrated in FIG. 7C to be displayed on the touch panel so as to accept a user setup inspection span T0y. The user touches fields of one or more of the numerical keys corresponding to a span that the user wants to set as a user setup inspection span T0y, and finally touches a field of an OK key. Through this operation, the user setup inspection span T0y is input into the jaundice meter A. Upon accepting the user setup inspection span T0y, the inspection span storage control section 371 operates to cause the user setup inspection span T0y to be stored in the inspection span storage section 361 as a user setup value. In this way, the user setup inspection span T0y is set in the jaundice meter A

On the other hand, in the above embodiment, when a user wants to set the calibration span D0, an operation therefor is performed, for example, in the following manner. In the example illustrated in FIG. 7A, a user touches the “setting” field in the main menu displayed on the touch panel, so that an instruction for start of the setting is input into the jaundice meter A, and thereby the jaundice meter A is operated in the setting mode.

In the setting mode, the inspection span storage control section 371 operates to cause the span setting sub-menu illustrated in FIG. 7B to be displayed. The user touches the “calibration span setting” field in the span setting sub-menu displayed on the touch panel, so that an instruction for inspection span setting is input into the jaundice meter A, and thereby the jaundice meter A is operated in the calibration span setting mode.

In the calibration span setting mode, the calibration span storage control section 372 operates to cause the numerical key set illustrated in FIG. 7C to be displayed on the touch panel so as to accept a user setup calibration span D0y. The user touches fields of one or more of the numerical keys corresponding to a span that the user wants to set as a user setup calibration span D0y, and finally touches the field of the OK key. Through this operation, the user setup calibration span D0y is input into the jaundice meter A. Upon accepting the user setup calibration span D0y, the calibration span storage control section 372 operates to cause the user setup calibration span D0y to be stored in the calibration span storage section 362 as a user setup value. In this way, the user setup calibration span D0y is set in the jaundice meter A

As described above, the jaundice meter A according to the above embodiment comprises, as one example of the warning control unit, the inspection warning output section 331, the inspection span elapse determination section 341, the inspection date storage section 351, the inspection span storage section 361 and the time measurement section 38. Thus, it becomes possible to warn of an inspection timing to inspect the measuring unit 1 and the signal processing unit 2 which are one example of the jaundice measurement unit.

In the jaundice meter A according to the above embodiment, a default value T0d of an inspection span T0 can be preliminarily stored in the inspection span storage section 361. Thus, a user can adequately use the jaundice meter A, without knowledge of an adequate inspection span T0.

The jaundice meter A according to the above embodiment comprises the inspection span input section 41 and the inspection span storage control section 371. Thus, a user can freely set the inspection span T0. For example, in a situation where a frequency of usage of the jaundice meter A is relatively small, an inspection span T0y can be set to a relatively large value. On the other hand, in a situation where the frequency of usage of the jaundice meter A is relatively large, the inspection span T0y can be set to a relatively small value.

The jaundice meter A according to the above embodiment comprises, as one example of the warning control unit, the calibration warning output section 332, the calibration span elapse determination section 342, the calibration date storage section 352, the calibration span storage section 362 and the time measurement section 38. Thus, it becomes possible to warn of a calibration timing to calibrate the measuring unit 1 and the signal processing unit 2 which are one example of the jaundice measurement unit.

In the jaundice meter A according to the above embodiment, a default value T0d of a calibration span D0 can be preliminarily stored in the calibration span storage section 362. Thus, a user can adequately use the jaundice meter A, without knowledge of an adequate calculation span D0.

The jaundice meter A according to the above embodiment comprises the calibration span input section 42 and the calibration span storage control section 372. Thus, a user can freely set the calibration span D0. For example, in a situation where a frequency of usage of the jaundice meter A is relatively small, a calibration span D0y can be set to a relatively large value. On the other hand, in a situation where the frequency of usage of the jaundice meter A is relatively large, the calibration span D0y can be set to a relatively small value.

In the jaundice meter A according to the above embodiment, the measuring unit 1 and the signal processing unit 2 are subjected to the inspection. In the jaundice meter A, particularly a decrease of light intensity causes difficulty in obtaining a normal measurement result. Thus, the jaundice meter A may be configured to inspect at least the measuring unit 1.

In the jaundice meter A according to the above embodiment, the measurement control section 31 may be configured to determine whether or not an instruction for execution of the inspection has been issued from the inspection execution control section 321 after the output of the inspection warning by the inspection warning output section 331, and stop an operation of the jaundice measurement unit (i.e., in the above embodiment, the measuring unit 1 and the signal processing unit 2), until completion of the inspection of a state of the jaundice measurement unit executed by the inspection execution control section 321. This makes it possible to perform measurement of the degree of jaundice using the jaundice measurement unit after reliably inspecting the jaundice measurement unit.

In the jaundice meter A according to the above embodiment, the measurement control section 31 may be configured to count the number of the inspection warnings output from the inspection warning output section 331, and stop the operation of the jaundice measurement unit during a period until completion of the inspection of a state of the jaundice measurement unit executed by the inspection execution control section 321 after a counted value of the number of the inspection warnings reaches a preset first number threshold Th1. This makes it possible to provide a time for allowing the measurement of the degree of jaundice using the jaundice measurement unit, before the inspection is actually executed after the inspection warning. Thus, for example, in a situation where it is necessary to urgently use the jaundice meter A, even when the inspection warming is output, the measurement of the degree of jaundice can be performed using the jaundice measurement unit, without performing the inspection.

In the jaundice meter A according to the above embodiment, the measurement control section 31 may be configured to determine whether or not an instruction for execution of the calibration has been issued from the calibration execution control section 322 after the output of the calibration warning by the calibration warning output section 332, and stop an operation of the jaundice measurement unit (i.e., in the above embodiment, the measuring unit 1 and the signal processing unit 2), until completion of the calibration of the jaundice measurement unit executed by the calibration execution control section 322. This makes it possible to perform measurement of the degree of jaundice using the jaundice measurement unit after reliably calibrating the jaundice measurement unit.

In the jaundice meter A according to the above embodiment, the measurement control section 31 may be configured to count the number of the calibration warnings output from the calibration warning output section 332, and stop the operation of the jaundice measurement unit during a period until completion of the calibration of the jaundice measurement unit executed by the calibration execution control section 322 after a counted value of the number of the calibration warnings reaches a preset second number threshold Th2. This makes it possible to provide a time for allowing the measurement of the degree of jaundice using the jaundice measurement unit, before the calibration is actually executed after the calibration warning. Thus, for example, in a situation where it is necessary to urgently use the jaundice meter A, even when the calibration warming is output, the measurement of the degree of jaundice can be performed using the jaundice measurement unit, without performing the calibration.

While this specification discloses various techniques as mentioned above, major ones of the techniques will be outlined below.

According one aspect of the present invention, there is provided a jaundice meter which comprises a jaundice measurement unit configured to measure a degree of jaundice by using light, and a warning control unit configured to recommend an inspection of the jaundice measurement unit. Preferably, the warning control unit comprises: a time measurement section configured to measure a time and date; an inspection date storage section configured to store therein an inspection date when the jaundice measurement unit is inspected; an inspection span storage section configured to store therein an inspection span which is a time interval at which the inspection is to be performed; an inspection span elapse determination section configured to determine whether or not the inspection span stored in the inspection span storage section has elapsed from most recent inspection date stored in the inspection date storage section; and an inspection warning output section configured to output an inspection warning for recommending the inspection of the jaundice measurement unit, when the inspection span elapse determination section determines that the inspection span has elapsed.

This jaundice meter comprises the warning control unit, so, that it becomes possible to warn of an inspection timing to inspect the jaundice measurement unit.

According to one specific embodiment, in the above jaundice meter, the inspection span storage section preliminarily stores therein a default value of the inspection span.

In this jaundice meter, a default value of the inspection span can be preliminarily stored in the inspection span storage section. Thus, a user can adequately use the jaundice meter, without knowledge of an adequate inspection span.

According to another specific embodiment, the above jaundice meter further comprises; an inspection span input section configured to allow the inspection span to be externally input therethrough; and an inspection span storage control section configured to cause the inspection span input through the inspection span input section to be stored in the inspection span storage section.

This jaundice meter comprises the inspection span input section and the inspection span storage control section. Thus, a user can freely set the inspection span. For example, in a situation where a frequency of usage of the jaundice meter is relatively small, the inspection span can be set to a relatively large value. On the other hand, in a situation where the frequency of usage of the jaundice meter is relatively large, the inspection span can be set to a relatively small value.

According to another specific embodiment, the above jaundice meter further comprises; an inspection execution section configured to execute the inspection of the jaundice measurement unit; and a first measurement control section configured to stop an operation of the jaundice measurement unit during a period until completion of the inspection of the jaundice measurement unit executed by the inspection execution section after the output of the inspection warning by the inspection warning output section.

This jaundice meter comprises the measurement control section, so that it becomes possible to perform measurement of the degree of jaundice using the jaundice measurement unit after reliably inspecting the jaundice measurement unit.

According to another specific embodiment, in the above jaundice meter, the first measurement control section is configured to count the number of the inspection warnings output from the inspection warning output section, and stop the operation of the jaundice measurement unit during a period until completion of the inspection of the jaundice measurement unit executed by the inspection execution section after a counted value of the number of the inspection warnings reaches a preset first number threshold.

This jaundice meter makes it possible to provide a time for allowing the measurement of the degree of jaundice using the jaundice measurement unit, before the inspection is actually executed after the inspection warning. Thus, for example, in a situation where it is necessary to urgently use the jaundice meter, even when the inspection warming is output, the measurement of the degree of jaundice can be performed using the jaundice measurement unit, without performing the inspection.

According to another specific embodiment, in the above jaundice meter, the warning control unit is configured to recommend calibration of the jaundice measurement unit depending on a result of the inspection of the jaundice measurement unit. Preferably, the warning control unit further comprises: a calibration date storage section configured to store therein calibration date when the jaundice measurement unit is calibrated; a calibration span storage section configured to store therein a calibration span which is a time interval at which the calibration is to be performed; a calibration span elapse determination section configured to determine whether or not the calibration span stored in the calibration span storage section has elapsed from most recent calibration date stored in the calibration date storage section; and a calibration warning output section configured to output a calibration warning for recommending the calibration of the jaundice measurement unit, when the calibration span elapse determination section determines that the calibration span has elapsed.

In this jaundice meter, the warning control output unit additionally outputs the calibration warning, so that it becomes possible to warn of a calibration timing to calibrate the jaundice measurement unit.

According to another specific embodiment, in the above jaundice meter, the calibration span storage section preliminarily stores therein a default value of the calibration span.

In this jaundice meter, a default value of the calibration span can be preliminarily stored in the calibration span storage section. Thus, a user can adequately use the jaundice meter, without knowledge of an adequate calibration span.

According to another specific embodiment, the above jaundice meter further comprises; a calibration span input section configured to allow the calibration span to be externally input therethrough; and a calibration span storage control section configured to cause the calibration span input through the calibration span input section to be stored in the calibration span storage section.

This jaundice meter comprises the calibration span input section and the calibration span storage control section. Thus, a user can freely set the calibration span. For example, in a situation where a frequency of usage of the jaundice meter is relatively small, the calibration span can be set to a relatively large value. On the other hand, in a situation where the frequency of usage of the jaundice meter is relatively large, the calibration span can be set to a relatively small value.

According to another specific embodiment, the above jaundice meter further comprises; a calibration execution section configured to execute the calibration of the jaundice measurement unit; and a second measurement control section configured to stop an operation of the jaundice measurement unit during a period until completion of the calibration of the jaundice measurement unit executed by the calibration execution section after the output of the calibration warning by the calibration warning output section.

This jaundice meter comprises the second measurement control section, so that it becomes possible to perform measurement of the degree of jaundice using the jaundice measurement unit after reliably calibrating the jaundice measurement unit.

According to another specific embodiment, in the above jaundice meter, the second measurement control section is configured to count the number of the calibration warnings output from the calibration warning output section, and stop the operation of the jaundice measurement unit during a period until completion of the calibration of the jaundice measurement unit executed by the calibration execution control section after a counted value of the number of the calibration warnings reaches a preset second number threshold.

This jaundice meter makes it possible to provide a time for allowing the measurement of the degree of jaundice using the jaundice measurement unit, before the calibration is actually executed after the calibration warning. Thus, for example, in a situation where it is necessary to urgently use the jaundice meter, even when the calibration warming is output, the measurement of the degree of jaundice can be performed using the jaundice measurement unit, without performing the calibration.

This application is based on Japanese Patent Application Serial No. 2012-148417 filed in Japan Patent Office on Jul. 2, 2012, the contents of which are hereby incorporated by reference.

Although the present invention has been adequately and fully described by way of example with reference to the accompanying drawings, it is to be understood that various changes and/or modifications will be apparent to those skilled in the art. Therefore, unless otherwise such changes and modifications depart from the scope of the present invention hereinafter defined, they should be construed as being included therein.

### INDUSTRIAL APPLICABILITY

The present invention can provide a jaundice meter.

1. A jaundice meter comprising:
a jaundice measurement unit configured to measure a degree of jaundice by using light; and
a warning control unit configured to recommend an inspection of the jaundice measurement unit.

2. The jaundice meter as defined in claim 1, wherein the warning control unit comprises:
a time measurement section configured to measure a time and date;
an inspection date storage section configured to store therein an inspection date when the jaundice measurement unit is inspected;
an inspection span storage section configured to store therein an inspection span which is a time interval at which the inspection is to be performed;
an inspection span elapse determination section configured to determine whether or not the inspection span stored in the inspection span storage section has elapsed from most recent inspection date stored in the inspection date storage section; and
an inspection warning output section configured to output an inspection warning for recommending the inspection of the jaundice measurement unit, when the inspection span elapse determination section
determines that the inspection span has elapsed.

3. The jaundice meter as defined in claim 2, wherein the inspection span storage section preliminarily stores therein a default value of the inspection span.

4. The jaundice meter as defined in claim 2, which further comprises;
an inspection span input section configured to allow the inspection span to be externally input therethrough; and
an inspection span storage control section configured to cause the inspection span input through the inspection span input section to be stored in the inspection span storage section.

5. The jaundice meter as defined in claim 2, which further comprises;
an inspection execution section configured to execute the inspection of the jaundice measurement unit; and
a first measurement control section configured to stop an operation of the jaundice measurement unit during a period until completion of the inspection of the jaundice measurement unit executed by the inspection execution section after the output of the inspection warning by the inspection warning output section.

6. The jaundice meter as defined in claim 5, wherein the first measurement control section is configured to count the number of the inspection warnings output from the inspection warning output section, and stop the operation of the jaundice measurement unit during a period after a counted value of the number of the inspection warnings reaches a preset first number threshold through until completion of the inspection of the jaundice measurement unit executed by the inspection execution section.

7. The jaundice meter as defined in claim 1, wherein the warning control unit is configured to recommend calibration of the jaundice measurement unit depending on a result of the inspection of the jaundice measurement unit.

8. The jaundice meter as defined in claim 2 wherein the warning control unit further comprises:
a calibration date storage section configured to store therein calibration date when the jaundice measurement unit is calibrated;
a calibration span storage section configured to store therein a calibration span which is a time interval at which the calibration is to be performed;
a calibration span elapse determination section configured to determine whether or not the calibration span stored in the calibration span storage section has elapsed from most recent calibration date stored in the calibration date storage section; and
a calibration warning output section configured to output a calibration warning for recommending the calibration of the jaundice measurement unit, when the calibration span elapse determination section
determines that the calibration span has elapsed.

9. The jaundice meter as defined in claim 8, wherein the calibration span storage section preliminarily stores therein a default value of the calibration span.

10. The jaundice meter as defined in claim 8, which further comprises;
a calibration span input section configured to allow the calibration span to be externally input therethrough; and
a calibration span storage control section configured to cause the calibration span input through the calibration span input section to be stored in the calibration span storage section.

11. The jaundice meter as defined in claim 8, which further comprises;
a calibration execution section configured to execute the calibration of the jaundice measurement unit; and
a second measurement control section configured to stop an operation of the jaundice measurement unit during a period after the output of the calibration warning by the calibration warning output section through until completion of the calibration of the jaundice measurement unit executed by the calibration execution section.

12. The jaundice meter as defined in claim 11, wherein the second measurement control section is configured to count the number of the calibration warnings output from the calibration warning output section, and stop the operation of the jaundice measurement unit during a period until completion of the calibration of the jaundice measurement unit executed by the calibration execution control section after a counted value of the number of the calibration warnings reaches a preset second number threshold.
